(19) United States
(12) Patent Application Publication
(10) Pub. No.: US 2025/0386722 A1
(43) Pub. Date: Dec. 18, 2025

(54) COMPOUND, POLYMER COMPOUND, PHOTOSENSITIVE SURFACE TREATMENT AGENT, LAMINATE, SUBSTRATE FOR PATTERN FORMATION, TRANSISTOR, PATTERN FORMATION METHOD, AND TRANSISTOR MANUFACTURING METHOD

(71) Applicant: NIKON CORPORATION, Tokyo (JP)

(72) Inventors: Yusuke KAWAKAMI, Yokohama-shi (JP); Kazuo YAMAGUCHI, Yokohama-shi (JP); Noriko CHIKARAISHI, Yokohama-shi (JP); Michiko ITOU, Yokohama-shi (JP)

(73) Assignee: NIKON CORPORATION, Tokyo (JP)

(21) Appl. No.: 19/247,268

(22) Filed: Jun. 24, 2025

Related U.S. Application Data
(63) Continuation of application No. PCT/JP2023/044418, filed on Dec. 12, 2023.

(30) Foreign Application Priority Data
Dec. 27, 2022 (JP) 2022-209535

(51) Int. Cl. H10K 71/00; C07C 271/12; C07C 271/22; C07F 7/18; C08F 20/36; C23C 18/16; G03F 7/027; G03F 7/038; H10K 10/46; H10K 10/84

(52) U.S. Cl. CPC H10K 71/621; C07C 271/12; C07C 271/22; C07F 7/1804; C08F 20/36; C23C 18/1605; G03F 7/027; G03F 7/0384; H10K 10/466; H10K 10/481; H10K 10/488; H10K 10/84

(57) ABSTRACT

A compound represented by General Formula (M1) below is provided. In Formula (M1), Y is a linear or branched alkyl group having 1 to 10 carbon atoms, a polymerizable group-containing group, or a group represented by [SiX₃—Y¹¹—*]. Y¹¹ is a linear or branched alkylene group having 1 to 4 carbon atoms, X is a halogen atom or an alkoxy group, and * is a bonding site to an N atom. R¹ is a hydrogen atom or a methyl group. R² is a hydrogen atom or a alkyl group having 1 to 6 carbon atoms. R³ and R⁴ each independently represents a alkyl group having 1 to 3 carbon atoms or a fluoroalkyl group. n=2.

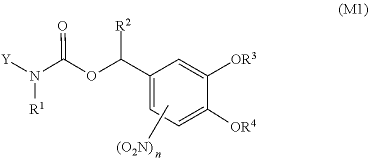

### CROSS-REFERENCE TO RELATED APPLICATIONS

The present application is a continuation application of International Application No. PCT/JP2023/044418, filed on Dec. 12, 2023, which claims priority to Japanese Patent Application No. 2022-209535, filed on Dec. 27, 2022, the contents of each of which are incorporated herein by reference.

### TECHNICAL FIELD

The present invention relates to a compound, a polymer compound, a photosensitive surface treatment agent, a laminate, a substrate for pattern formation, a transistor, a pattern formation method, and a transistor manufacturing method.

### BACKGROUND ART

In recent years, in the manufacture of microdevices such as semiconductor elements, integrated circuits, and organic EL display devices, a method has been proposed for forming patterns with different surface properties on a substrate and utilizing these differences in surface properties to fabricate microdevices.

As an example of a pattern formation method that utilizes differences in surface properties on a substrate, there is a method for forming a region in which chemically active substituents are generated in part of the substrate. This method allows a metal material, an organic material, or an inorganic material to adhere closely to the part of the substrate.

Electroless plating is a technique for making a metal material adhere closely onto a substrate to form a metal film. For example, Patent Document 1 discloses a technique for forming fine wiring by electroless plating. Specifically, Patent Document 1 discloses that a catalytic activation layer and a photoresist are used to perform photopatterning by etching or lift-off after plating one surface.

### CITATION LIST

### Patent Document

Patent Document 1: Japanese Unexamined Patent Application, First Publication No. 2006-2201

### SUMMARY OF INVENTION

One aspect of the present invention is a compound represented by General Formula (M1) below.

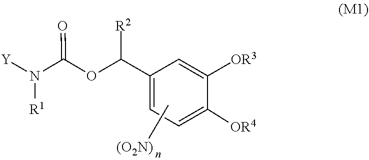

(In Formula (M1), Y is a linear or branched alkyl group having 1 to 10 carbon atoms, a polymerizable group-containing group, or a group represented by [SiX₃—Y¹¹—*], Y¹¹ is a linear or branched alkylene group having 1 to 4 carbon atoms, X is a halogen atom or an alkoxy group, * is a bonding site to an N atom, R¹ is a hydrogen atom or a methyl group, R² is a hydrogen atom or an alkyl group having 1 to 6 carbon atoms, R³ and R⁴ each independently represents an alkyl group having 1 to 3 carbon atoms or a fluoroalkyl group having 1 to 3 carbon atoms, and n=2.)

### BRIEF DESCRIPTION OF DRAWINGS

FIG. 1 A schematic view explaining a pattern formation method of the present embodiment.

FIG. 2 A schematic view explaining a transistor manufacturing method according to the present embodiment.

### DESCRIPTION OF EMBODIMENT

### <Compound>

One aspect of the present invention is a compound represented by General Formula (M1) below.

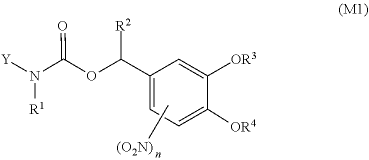

In Formula (M1), Y is a linear or branched alkyl group having 1 to 10 carbon atoms, and the alkyl group for Y is preferably a linear or branched alkyl group having 1 to 5 carbon atoms. Specific examples of Y include a methyl group, an ethyl group, a propyl group, an isopropyl group, an n-butyl group, an isobutyl group, a tert-butyl group, a pentyl group, an isopentyl group, and a neopentyl group.

In Formula (M1), examples of a polymerizable group-containing group for Y include a group represented by [CH₂═(C—R¹¹)—C(═O)—O—(Y¹¹)—*]. R¹¹ is a hydrogen atom or a methyl group, Y¹¹ is a linear or branched alkylene group having 1 to 4 carbon atoms, and * is a bonding site to an N atom.

In Formula (M1), when Y is a group represented by “SiX₃—Y¹¹—*,” X is a halogen atom or an alkoxy group. Examples of halogen atoms represented by X include a fluorine atom, a chlorine atom, a bromine atom, and an iodine atom.

Examples of Y¹¹ include a methylene group [—CH₂—], an ethylene group [—(CH₂)₂—], a trimethylene group [—(CH₂)₃—], and a tetramethylene [—(CH₂)₄—]. In addition, examples of Y¹ include —CH(CH₃)—, —CH(CH₂CH₃)—, —C(CH₃)₂—, and —C(CH₃) (CH₂CH₃)—.

X is preferably an alkoxy group. Examples of alkoxy groups for X include —O—(CH₃) and —O—(CH₂)n12 (CH₃). n12 is a natural number of 1 to 3.

In Formula (M1), R¹ is a hydrogen atom or a methyl group.

In Formula (M1), R² is a hydrogen atom or an alkyl group having 1 to 6 carbon atoms. The alkyl group for R² is preferably an alkyl group having 1 to 3 carbon atoms. A methyl group, an ethyl group, and an isopropyl group are preferable, and an isopropyl group is more preferable.

In Formula (M1), R³ and R⁴ each independently represents an alkyl group having 1 to 3 carbon atoms or a fluoroalkyl group having 1 to 3 carbon atoms.

The alkyl group for R³ and R⁴ is preferably an linear or branched alkyl group having 1 to 3 carbon atoms, and specific examples thereof include a methyl group, an ethyl group, and a propyl group.

When R³ and R⁴ are fluoroalkyl groups, they may be linear or branched alkyl groups having 1 to 3 carbon atoms which have been partially fluorinated, or they may be perfluoroalkoxy groups. In the present embodiment, fluorinated alkoxy groups which have been partially fluorinated are preferable.

n=2. In Formula (M1),

The compound represented by Formula (M1) has a dinitrobenzyl group where n=2. When the compound represented by Formula (M1) is irradiated with light, the dinitrobenzyl group is eliminated to generate an amine. A metal material, an organic material, or an inorganic material can adhere closely to the moiety where the amine is generated. The amine generated from the compound represented by Formula (M1) is a primary amine (—NH₂) or a secondary amine (—NH—).

The compound represented by Formula (M1) has a higher photoreaction efficiency (Φ365) than a compound having a mononitrobenzyl group. The photoreaction efficiency (Φ365) in the present specification is a ratio of a photodecomposition rate constant (k) to an absorbance (A365), and is an index showing the efficiency of a reaction to absorbed light. Specifically, the photoreaction efficiency (Φ) is calculated by the following equation.

Photoreaction efficiency (Φ365)=photodecomposition rate constant (k)/absorbance (A365)

The larger the value of the photoreaction efficiency (Φ365) obtained by the above-described equation, the higher the efficiency of the reaction to the received light, meaning that the elimination reaction of the protecting group is likely to proceed even with a low exposure dose.

According to the studies of the present inventors, it was found that, in the compound represented by Formula (M1), a compound having a dinitrobenzyl group has a higher photoreaction efficiency than a compound having a mononitrobenzyl group.

This is thought to be because an increase in the number of nitro groups or a structural change enhances the effect of the nitro groups contributing to the photoreaction. In this chemical structure, when exposed to light, a 6-membered ring transition state containing oxygen atoms of a nitro group is formed from hydrogen atoms at the benzyl position, and the hydrogens at the benzyl position migrate to the oxygens of the nitro group, which is the first step in the photodecomposition. To obtain the effect of the nitro group, the positional relationship with the hydrogens at the benzyl position and three-dimensional conformation are important. The improved photoreaction efficiency compared to that of the mononitrobenzyl group is inferred to be due to the fact that the compound containing a dinitrobenzyl group has an effective conformation for hydrogen migration.

Furthermore, according to the studies of the present inventors, it was found that, in the compound represented by Formula (M1), a compound having a dinitrobenzyl group has an improved photodecomposition rate compared to a compound having a mononitrobenzyl group.

Formula (M1) is preferably any one of the following Formulae (M1)-1, (M1)-2, and (M1)-3.

In the following formulae (M1)-1, (M1)-2, and (M1)-3, the explanations of symbols are the same as those in Formula (M1) above.

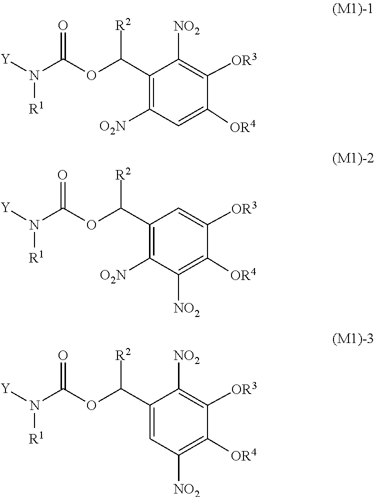

Specific examples of the compound represented by Formula (M1) will be shown below.

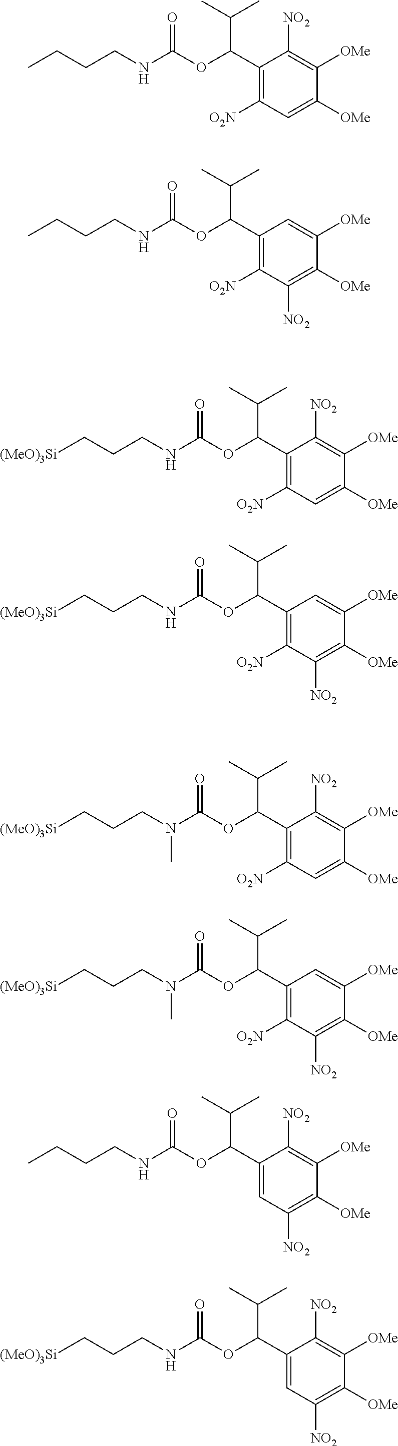

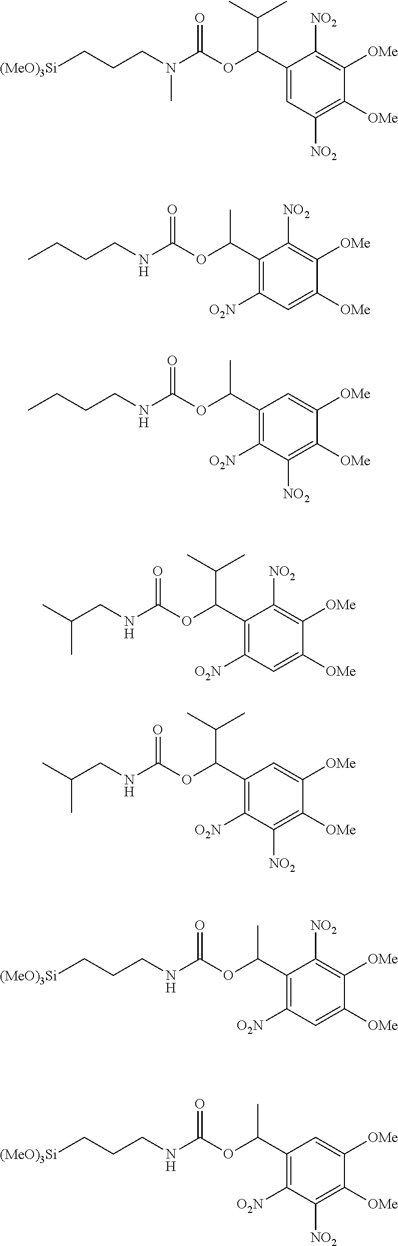

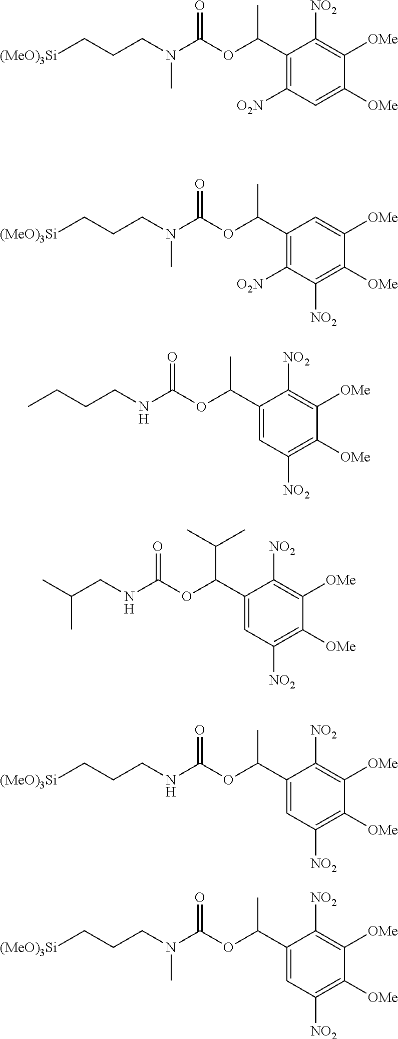

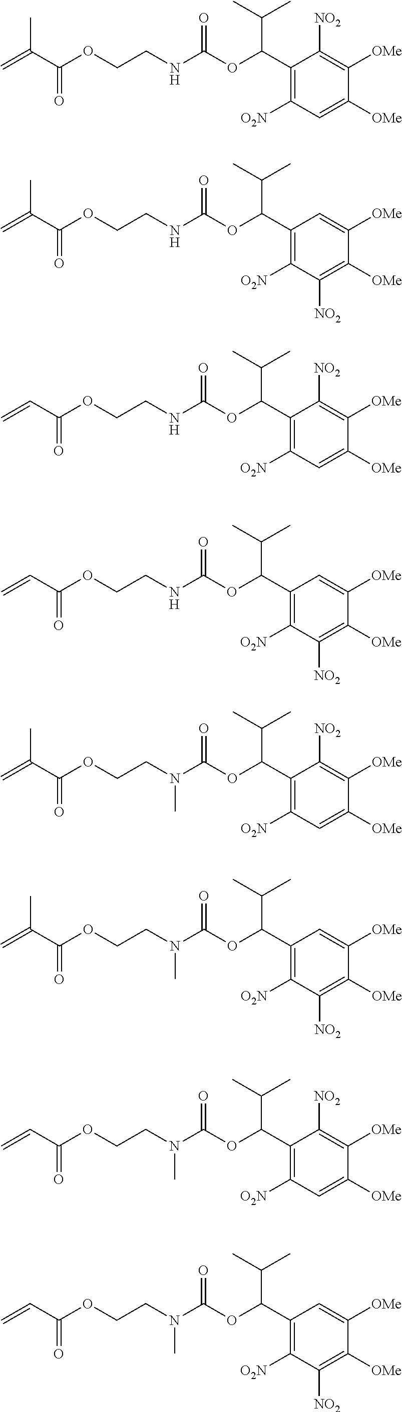

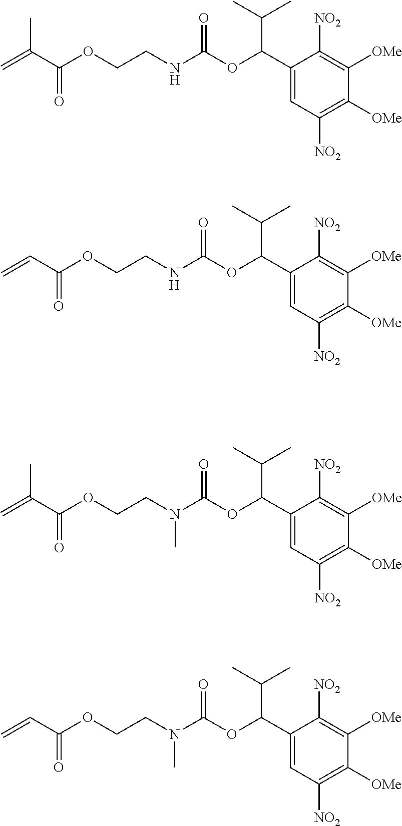 [SMILES: C=C(C)C(=O)OCCNC(=O)OC(c1cc([N+](=O)[O-])c(OC)c(OC)c1[N+](=O)[O-])C(C)C ; C=CC(=O)OCCNC(=O)OC(c1cc([N+](=O)[O-])c(OC)c(OC)c1[N+](=O)[O-])C(C)C ; C=C(C)C(=O)OCCN(C)C(=O)OC(c1cc([N+](=O)[O-])c(OC)c(OC)c1[N+](=O)[O-])C(C)C ; C=CC(=O)OCCN(C)C(=O)OC(c1cc([N+](=O)[O-])c(OC)c(OC)c1[N+](=O)[O-])C(C)C]

### <Method for Producing Compound (M1)>

The compound represented by Formula (M1) can be produced by the following method.

In the following description of the production method, n=2, and the explanation of each symbol is the same as that in Formula (M1) above.

The compound represented by Formula (M1) can be produced by a step of producing a dinitro intermediate 1 or a dinitro intermediate 2, followed by introduction of Y into the resulting dinitro intermediate 1 or dinitro intermediate 2.

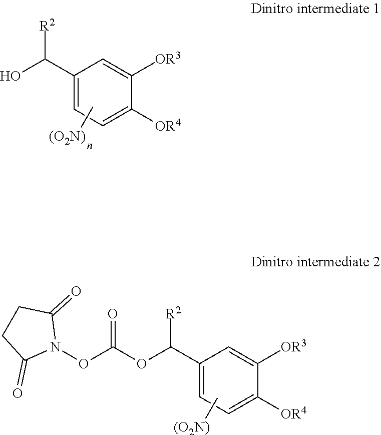

The dinitro intermediate 1 can be obtained by the reaction shown in (R)-1 below.

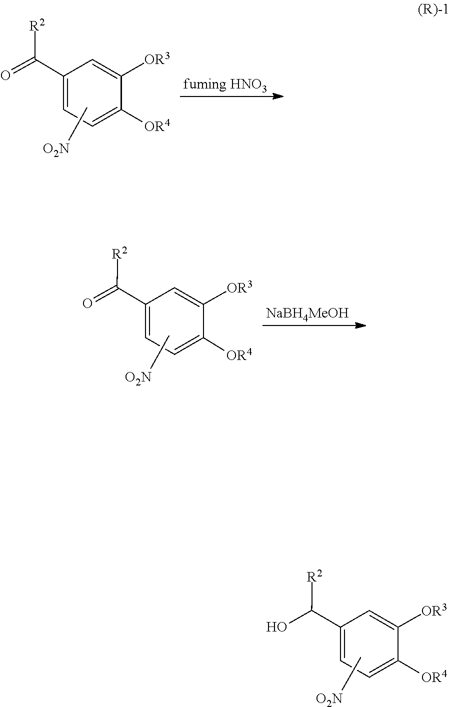

The dinitro intermediate 2 can be obtained by the reaction shown in (R)-2 below.

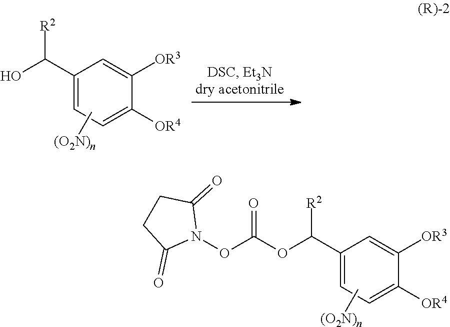

A reaction example in which Y is introduced into the resulting dinitro intermediate 1 or 2 to produce a compound (M1) will be shown below.

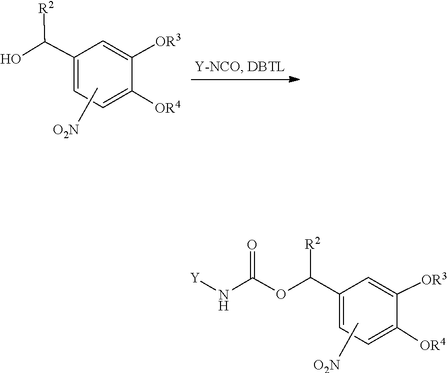

In the formula, “Y—NCO” is an alkyl isocyanate. An alkyl group in the alkyl isocyanate is preferably a linear or branched alkyl group having 1 to 5 carbon atoms, and specific examples thereof include a methyl group, an ethyl group, a propyl group, an isopropyl group, an n-butyl group, an isobutyl group, a tert-butyl group, a pentyl group, an isopentyl group, and a neopentyl group.

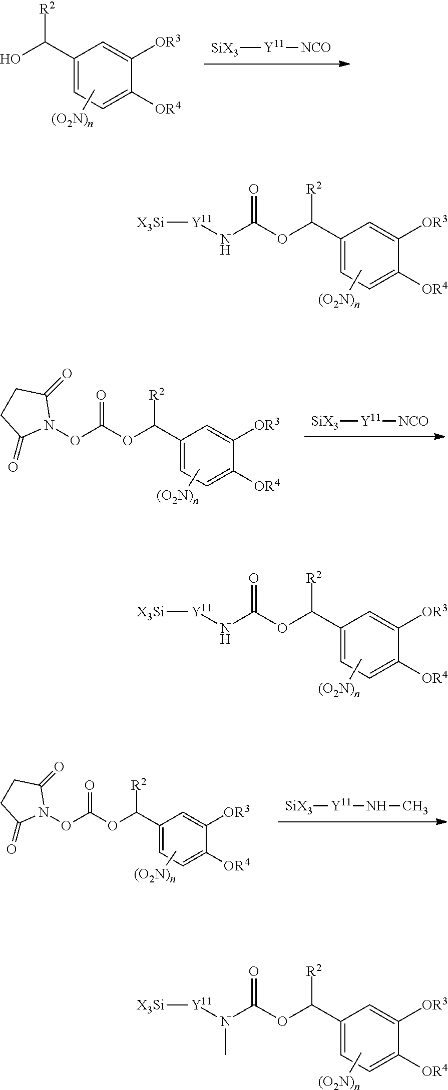

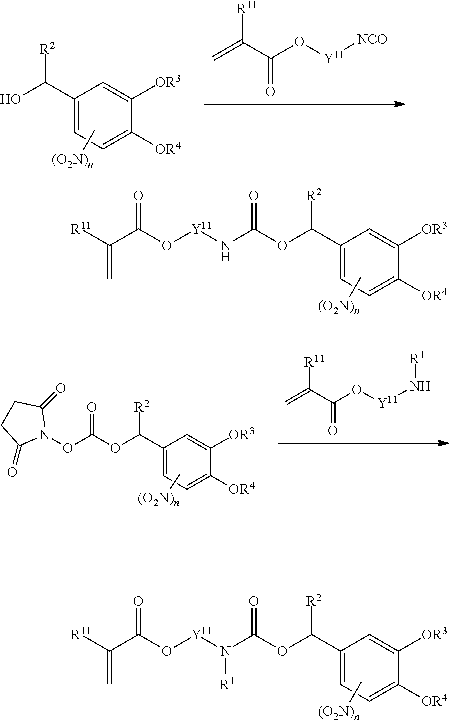

### <Method for Producing Compound (M1)-1>

A compound represented by Formula (M1)-1 can be produced by a step of producing a dinitro intermediate 1-1 or a dinitro intermediate 2-1, followed by introduction of Y into the resulting dinitro intermediate 1-1 or dinitro intermediate 2-1.

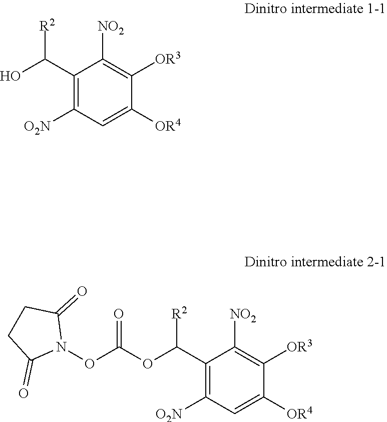

The dinitro intermediate 1-1 can be obtained by the reaction shown in (R)-1-1 below.

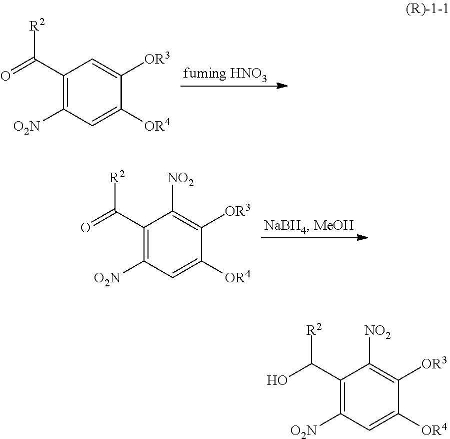

The dinitro intermediate 2-1 can be obtained by the reaction shown in (R)-2-1 below:

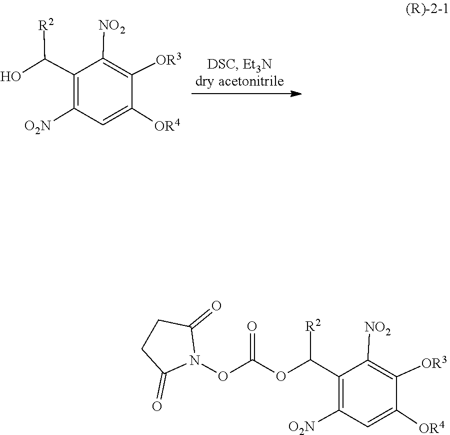

A reaction example in which Y is introduced into the resulting dinitro intermediate 1-1 or 2-1 to produce a compound (M1)-1 will be shown below:

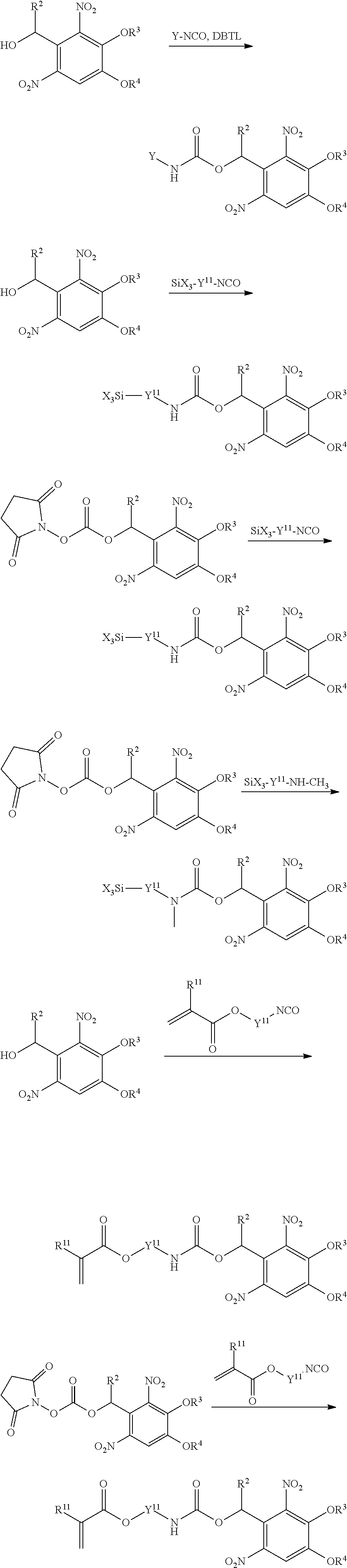

### <Method for Producing Compound (M1)-2>

A compound represented by Formula (M1)-2 can be produced by a step of producing a dinitro intermediate 1-2 or a dinitro intermediate 2-2, followed by introduction of Y into the resulting dinitro intermediate 1-2 or dinitro intermediate 2-2.

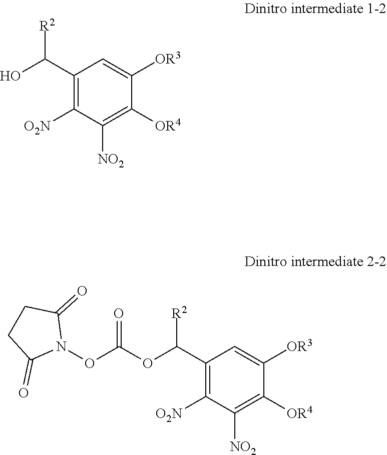

The dinitro intermediate 1-2 can be obtained by the reaction shown in (R)-1-2 below.

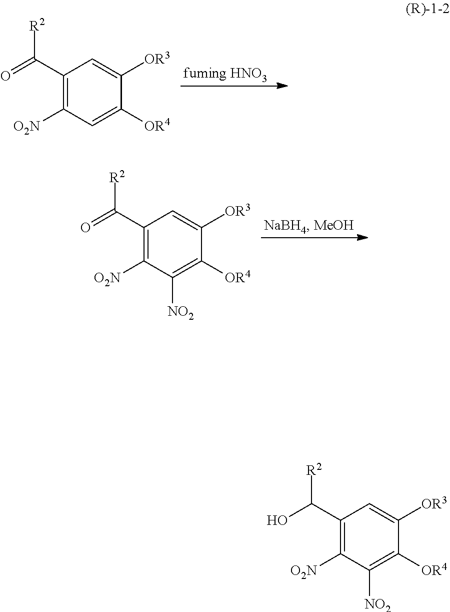

In the reaction shown in (R)-1-1 above and the reaction shown in (R)-1-2 above, dinitro intermediates 1-1 and 1-2 are both produced simultaneously. The products containing the dinitro intermediates 1-1 and 1-2 can be purified by silica gel column chromatography to obtain dinitro intermediates 1-1 and 1-2, respectively.

The dinitro intermediate 2-2 can be obtained by the reaction shown in (R)-2-2 below.

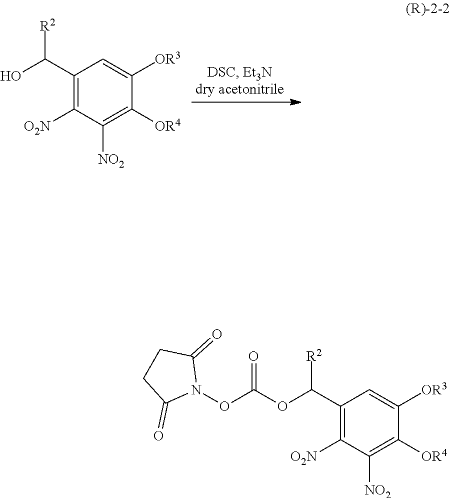

A reaction example in which Y is introduced into the resulting dinitro intermediate 1-2 or 2-2 to produce a compound (M1)-2 will be shown below.

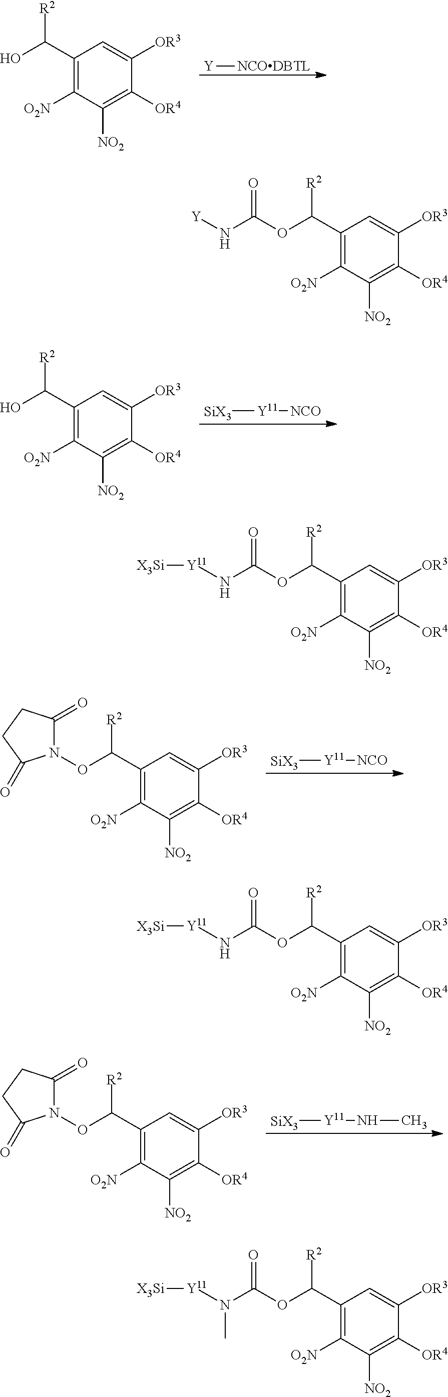

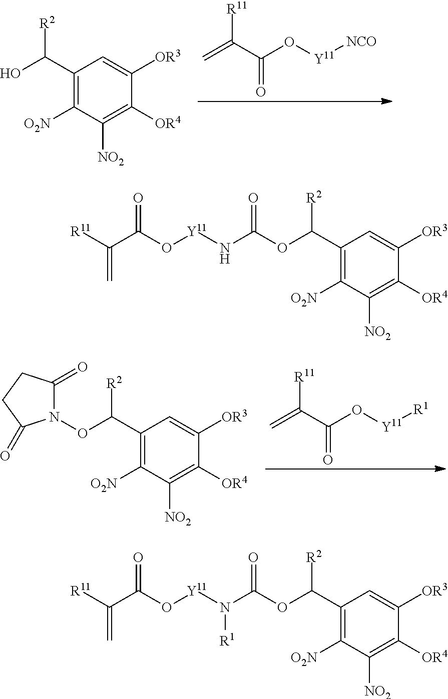

### <Polymer Compound>

One aspect of the present invention is a polymer compound having a repeating unit represented by Formula (P1) below:

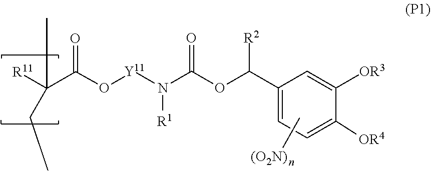

(In Formula (P1), R¹¹ is a hydrogen atom or a methyl group, and Y¹¹ is a linear or branched alkylene group having 1 to 4 carbon atoms, R¹ is a hydrogen atom or a methyl group, R² is a hydrogen atom or an alkyl group having 1 to 6 carbon atoms, R³ and R⁴ each independently represents an alkyl group having 1 to 3 carbon atoms or a fluoroalkyl group, and n=2.)

In Formula (P1), the explanations of R¹¹, Y¹¹, R¹, R³, and R⁴ are the same as those in Formula (M1).

The polymer compound having the repeating unit represented by Formula (P1) preferably has a substituent represented by Formula (1x) below bound to at least one end of the main chain. In Formula (1x) below, * means a bonding site with the end of the main chain of the polymer compound having the repeating unit represented by Formula (P1).

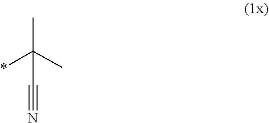

A polymer compound (P1)-A in which the substituent represented by Formula (1x) above is bound to the end of the main chain of the polymer compound having the repeating unit represented by Formula (P1) is exemplified below:

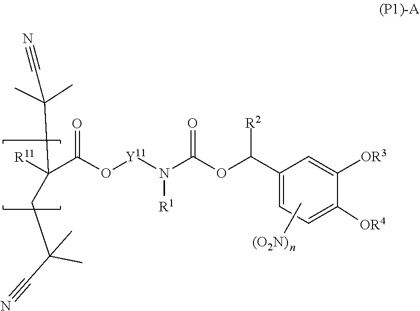

The repeating unit represented by Formula (P1) is preferably any one of Formulae (P1)-1 to (P1)-3 below:

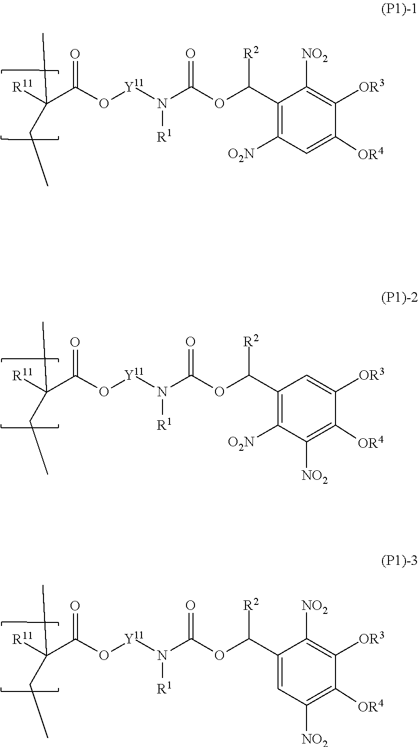

### <Method for Producing Polymer Compound (P1)>

A polymer compound having a repeating unit represented by Formula (P1) can be obtained by reacting a compound represented by Formula (M1) which contains a polymerizable group with various polymerization initiators such as a radical polymerization initiator and an anionic polymerization initiator. Examples of this reaction will be shown below.

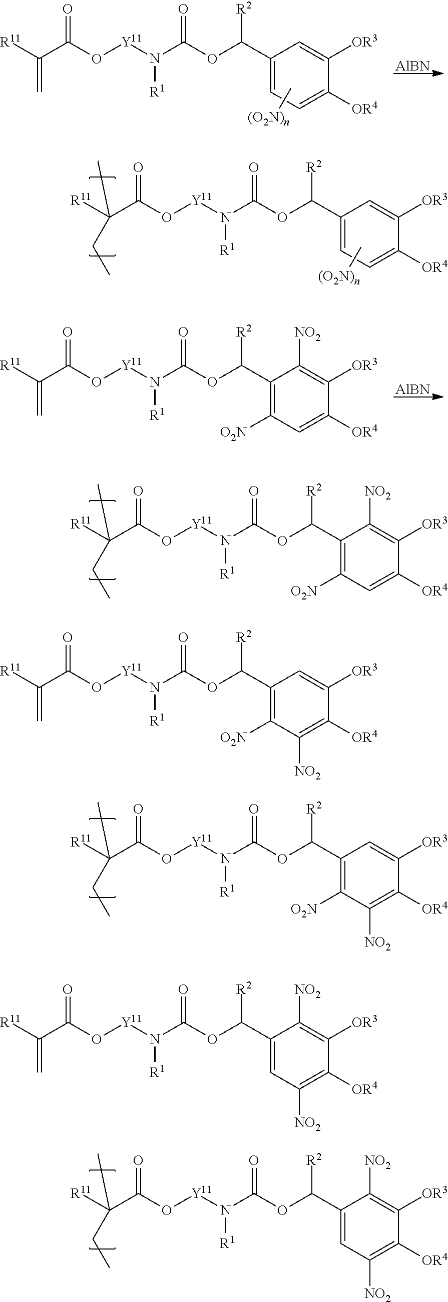

Polymerization of the compound represented by Formula (M1) which contains a polymerizable group is not particularly limited, and the compound may be polymerized by radical polymerization, anionic polymerization, or the like. Among these, a radical polymerization method is preferable from the viewpoint of ease of control or the like. From the viewpoint of obtaining desired solubility by controlling the molecular weight, controlled radical polymerization is more preferable among the radical polymerization methods.

Examples of the controlled radical polymerization method include a chain transfer agent method and a living radical polymerization method, which is a type of living polymerization. Living radical polymerization which facilitates control of molecular weight distribution is more preferable. Examples of living radical polymerization methods include a nitroxide radical polymerization method (NMP), an atom transfer radical polymerization method (ATRP), and a reversible addition-fragmentation chain transfer method (RAFT). From the viewpoints of temperature and versatility, an atom transfer radical polymerization method (ATRP) is particularly preferable.

Radical polymerization involving a chain transfer reaction is preferable from the viewpoints of productivity and economic efficiency, as well as from the viewpoint of obtaining desired film formability by broadening the molecular weight distribution from low to high molecular weights.

In a case of using radical polymerization, a conventionally known polymerization initiator can be appropriately used. In addition, a radical polymerization initiator may be used alone or in combination of two or more thereof, or commercially available products may be used as they are.

For example, an azo polymerization initiator which is a compound having an azo group (—N═N—) and generating N2 and radicals can be used. Specific examples thereof include azonitriles, azo esters, azo amides, azo amidines, and azo imidazolines. More specific examples thereof include 2,2′-azobis(2-amidinopropane) dihydrochloride, 4,4′-azobis(4-cyanovaleric acid), 2,2′-bis(2-imidazoline-2-yl)-2,2′-azopropane dihydrochloride, 2,2′-bis(2-imidazoline-2-yl)-2,2′-azopropane, 2,2′-azobis [N-(2-hydroxyethyl)-2-methylpropionamide], 2,2′-azobisisobutyronitrile (AIBN), and 2,2′-azobis(2,4-dimethylvaleronitrile) (ADVN).

Among these, 2,2′-azobisisobutyronitrile (AIBN) and 2,2′-azobis(2,4-dimethylvaleronitrile) (ADVN) are preferable, and 2,2′-azobisisobutyronitrile (AIBN) is particularly preferable.

The polymer compound of the present embodiment may consist of the repeating unit represented by Formula (P1), or may be a copolymer having, in addition to the repeating unit represented by Formula (P1), other repeating units as necessary. In the case where the polymer compound of the present embodiment is a copolymer having, in addition to the repeating unit represented by Formula (P1), other repeating units, the proportion of other such repeating units is, for example, 50 mol % or less, 40% % or less, or 30 mol % or less based on the total amount of all repeating units constituting the polymer compound (100 mol %).

Examples of other repeating units include methyl acrylate, phenyl acrylate, benzyl acrylate, dimethoxynitrobenzylcarbonylaminoethyl acrylate, fluorenylmethoxycarbonylaminoethyl acrylate, methyl methacrylate, phenyl methacrylate, benzyl methacrylate, dimethoxynitrobenzylcarbonylaminoethyl methacrylate, and fluorenylmethoxycarbonylaminoethyl methacrylate.

From the viewpoints of reducing the risk of dissolution and peeling in a plating bath or the like and ensuring solubility during film formation, the number-average molecular weight of the polymer compound having the repeating unit represented by Formula (P1) is preferably 300 to 100,000, which is sufficient for wet film formation, more preferably 1,000 to 90,000, and still more preferably 2,000 to 40,000. In addition, for the same reason, the peak of the molecular weight distribution is 1,000 to 90,000, and more preferably within a range of 2,000 to 40,000. These can be measured by gel permeation chromatography (GPC).

### <Photosensitive Surface Treatment Agent>

In one aspect of the present invention, a photosensitive surface treatment agent contains the compound represented by Formula (M1) above. In addition, in one aspect of the present invention, a photosensitive surface treatment agent may consist of the compound represented by Formula (M1) above.

In one aspect of the present invention, a photosensitive surface treatment agent contains the polymer compound having the repeating unit represented by Formula (P1) above. In addition, in one aspect of the present invention, a photosensitive surface treatment agent may consist of the polymer compound having the repeating unit represented by Formula (P1) above.

In one aspect of the present invention, a photosensitive surface treatment agent contains the compound represented by Formula (M1) above and the polymer compound having the repeating unit represented by Formula (P1) above.

In one aspect of the present invention, a photosensitive surface treatment agent may contain a solvent.

By dissolving it in common organic solvents such as alcohol-based solvents, ester-based solvents, hydrocarbon-based aromatic solvents, amine-based solvents, ketone-based solvents, glycol ether-based solvents, and ether-based solvents as solvents, it can be suitably used as a surface treatment agent.

Examples of alcohol-based solvents include isopropyl alcohol (IPA) and n-butyl alcohol (n-butanol).

Examples of ester-based solvents include ethyl acetate (EAC), butyl acetate (NBAC), n-propyl acetate (NPAC), and 3-methoxy-3-methylbutyl acetate.

Examples of the hydrocarbon-based aromatic solvents include toluene, xylene, benzene, ethylbenzene, and trimethylbenzene.

Examples of the amine-based solvents include N-methyl-2-pyrrolidone (NMP), dimethylformamide (DMF), and N,N-dimethylacetamide (DMAC).

Examples of ketone-based solvents include methyl ethyl ketone (MEK), methyl isobutyl ketone (MIBK), diisobutyl ketone (DIBK), methyl isopropyl ketone (MIPK), cyclohexanone, cyclopentanone (CPN), cycloheptanone, and acetone.

Examples of glycol ether-based solvents include methyl cellosolve, butyl cellosolve, ethylene glycol mono-t-butyl ether (ETB), propylene glycol monomethyl ether (PGME), propylene glycol monomethyl ether acetate (PGMEA), and 3-methoxy-3-methyl-1-butanol (MMB).

Examples of other solvents include halogen-based solvents containing chlorine or fluorine, and examples thereof include chloroform, chlorobenzene, and fluoroalkyl ethers. These may be used alone or in combination of two or more thereof. The organic solvents can be appropriately selected depending on conditions such as pollution-related properties, solubility, volatility, aggressiveness toward a substrate or an underlayer, film formation devices, and film formation methods.

In the present invention, by making the photosensitive surface treatment layer poorly soluble, it becomes insoluble in, for example, cleaning solutions, plating solutions, and solvents used in multilayer film formation, and wash resistance, process durability, and the like can be improved in wiring and lamination processes.

In the case of the photosensitive surface treatment agent consisting of the compound represented by Formula (M1) above, from the viewpoint of enhancing the reactivity between an undercoat and the photosensitive surface treatment agent and between molecules of the photosensitive surface treatment agent, as solvents contained therein, alcohol-based, ether-based, or hydrocarbon-based solvents are preferable, and hydrocarbon-based solvents are particularly preferable, with toluene being the most preferred.

Furthermore, from the viewpoint of enhancing the above-described reactivity, any acidic or basic compounds may be incorporated during film formation. These compounds can be appropriately selected according to the film formation conditions. Acidic compounds such as hydrochloric acid, acetic acid, and nitric acid are particularly preferable, with acetic acid being the most preferred.

In the case of the photosensitive surface treatment agent consisting of the polymer compound having the repeating unit represented by Formula (P1) above, from the viewpoints of solubility and film formability, as solvents contained therein, ester-based and ketone-based solvents are preferable, and ketone-based solvents are particularly preferable, with cyclopentanone being the most preferred.

The concentration of the compound represented by Formula (M1) above or the polymer compound having a repeating unit represented by Formula (P1) above contained in the photosensitive surface treatment agent can be appropriately selected according to the film formation conditions. From the viewpoints of storage stability and economic efficiency, the concentration thereof is preferably 0.001 to 10 mass %, more preferably 0.01 to 2 mass %, and among these, 0.1 to 0.3 mass % is preferred.

### <Pattern Formation Method>

A pattern formation method of the present embodiment includes a step of applying the photosensitive surface treatment agent of the present embodiment onto a substrate to form a photosensitive resin film: a step of irradiating the photosensitive resin film with light having a predetermined pattern to form an amine-generated region in an exposure region; and a step of placing an electroless plating catalyst in the amine-generated region to perform electroless plating.

Each step will be described below with reference to the drawings.

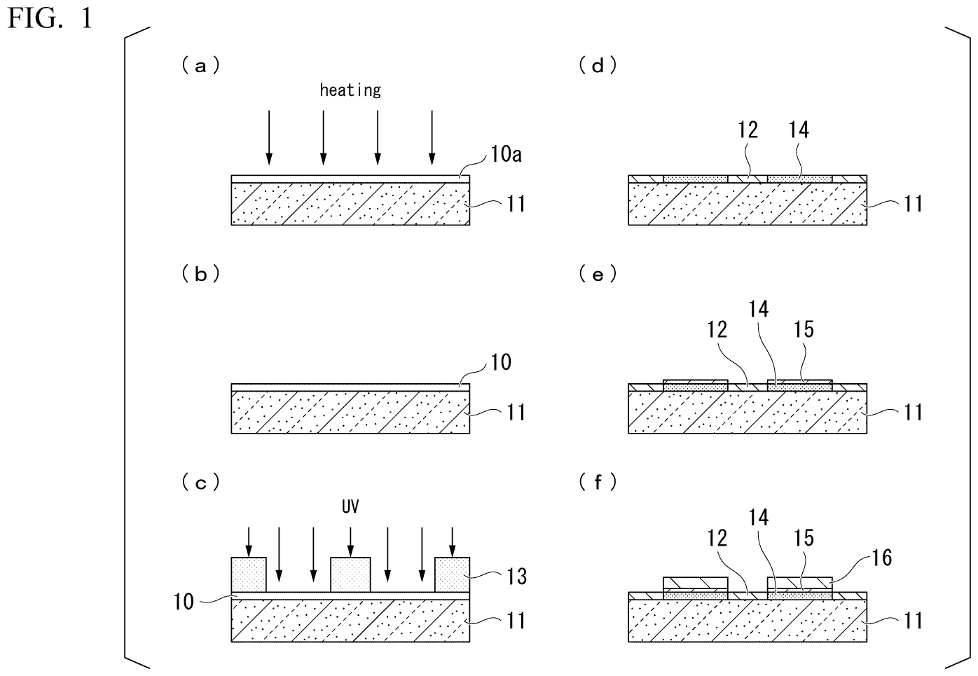

As shown in FIG. 1(a), a photosensitive surface treatment agent 10a of the present embodiment is applied onto a substrate 11.

Examples of application methods used include a spin coating method, a dip coating method, a die coating method, a spray coating method, a roll coating method, a microgravure method, a lip coating method, an ink jet method, applicator coating, and brush coating. In addition, the coating may be performed by a printing method such as flexographic printing or screen printing. In addition, the photosensitive surface treatment agent 10a may be used as a SAM film or an LB film.

In this step, as shown in FIG. 1(a), a treatment for drying the solvent by, for example, heat or reduced pressure may be included.

As a result, a photosensitive surface treatment agent layer 10 is formed on the substrate 11 as shown in FIG. 1(b).

Next, as shown in FIG. 1(c), a photomask 13 having an exposure region with a predetermined pattern is prepared. The exposure method is not limited to the method using a photomask, but methods such as projection exposure using an optical system such as lenses or mirrors, or maskless exposure using a spatial light modulator or a laser beam can be used. The photomask 13 may be provided so as to be in contact with the photosensitive surface treatment agent layer 10 or may be provided so as not to be in contact with the photosensitive surface treatment agent layer 10.

Thereafter, as shown in FIG. 1(c), the photosensitive surface treatment agent layer 10 is irradiated with UV light through the photomask 13. As a result, the photosensitive surface treatment agent layer 10 is exposed in an exposed region of the photomask 13.

As a result, as shown in FIG. 1(d), an amine-generated portion 14 is formed in an exposed portion, and an amine-non-generated portion 12 is formed in an unexposed portion.

Examples of UV light include an i-line with a wavelength of 365 nm. In addition, regarding the exposure dose or exposure time, deprotection does not need to progress completely, and it is sufficient if only a partial amount of amine is generated.

Next, as shown in FIG. 1(e), an electroless plating catalyst is applied to the surface to form a catalyst layer 15. The electroless plating catalyst is a catalyst that reduces metal ions contained in a plating solution for electroless plating, and examples thereof include silver and palladium.

Amino groups are exposed on the surface of the amine-generated portion 14. The amino groups are capable of capturing and reducing the above-described electroless plating catalyst. Therefore, the electroless plating catalyst is captured only on the amine-generated portion 14, forming the catalyst layer 15. In addition, an electroless plating catalyst that can be carried by amino groups can be used.

As shown in FIG. 1(f), electroless plating is performed to form a plating layer 16. Examples of materials for the plating layer 16 include nickel-phosphorus (Ni—P), gold (Au), and copper (Cu).

In this step, the substrate 11 is immersed in an electroless plating bath to reduce metal ions on the catalyst surface, thereby depositing the plating layer 16. At this time, since the catalyst layer 15 carrying a sufficient amount of catalyst is formed on the surface of the amine-generated portion 14, the plating layer 16 can be selectively deposited only on the amine-generated portion 14.

By the above-described steps, it is possible to form a wiring pattern on a predetermined substrate using the photosensitive surface treatment agent of the present embodiment.

### <Transistor Manufacturing Method>

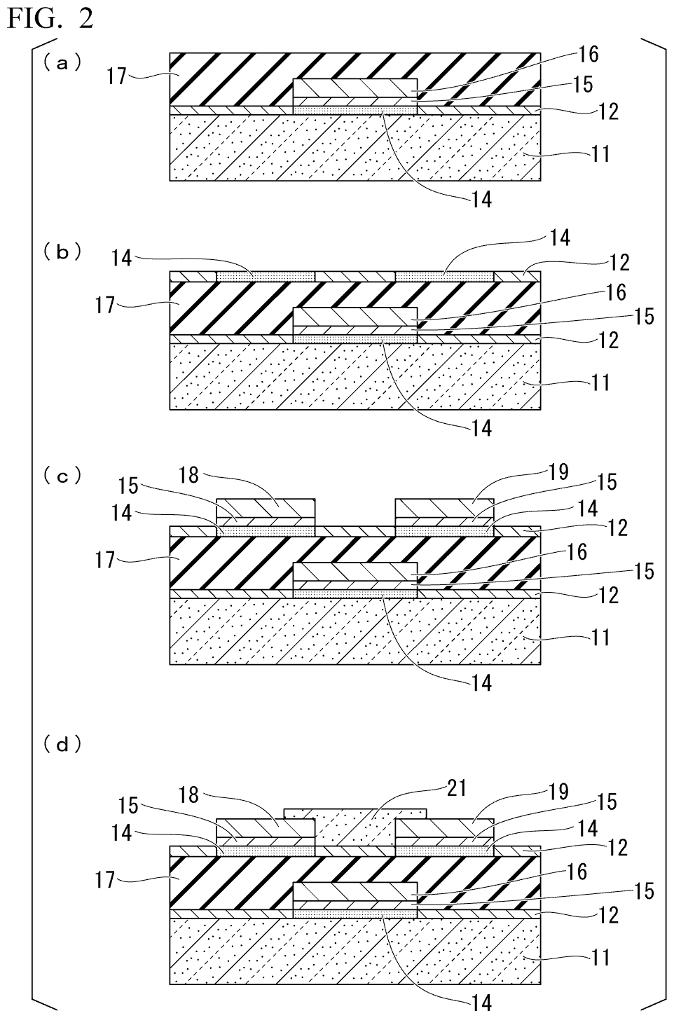

Furthermore, a transistor manufacturing method in which the plating layer 16 obtained in the above-described <Pattern formation method> is used as a gate electrode will be described with reference to FIG. 2.

As shown in FIG. 2(a), an insulator layer 17 is formed by a well-known method so as to cover the amine-non-generated portion 12 and the plating layer 16 of the electroless plating pattern formed by the above-described pattern formation method. The insulator layer 17 may be formed by applying a coating solution prepared by dissolving one or more resins, such as ultraviolet-curable acrylic resin, epoxy resin, ene-thiol resin, and silicone resin, in an organic solvent. By irradiating the coating film with ultraviolet light through a mask having an opening portion corresponding to the region where the insulator layer 17 is to be formed, the insulator layer 17 can be formed into a desired pattern. Before forming the insulator layer 17, the amine-non-generated portion 12 may be removed as necessary.

As shown in FIG. 2(b), in the same manner as in the above-described electroless plating pattern formation method, a photosensitive surface treatment agent layer 10 is formed on the insulator layer 17, and amine-generated portions 14 are formed in portions where a source electrode and a drain electrode are to be formed.

As shown in FIG. 2(c), in the same manner as in the above-described pattern formation method, an electroless plating catalyst is carried on the amine-generated portions 14 to form catalyst layers 15, and then electroless plating is performed to form a plating layer 18 (source electrode) and a plating layer 19 (drain electrode). Examples of materials for the plating layers 18 and 19 also include nickel-phosphorus (Ni—P) and copper (Cu). However, the plating layers 18 and 19 may be made of materials different from that of the plating layer 16 (gate electrode), or a different metal, for example, gold (Au), may be deposited on the surface of nickel-phosphorus (Ni—P) or copper (Cu) by performing electroless gold plating.

As shown in FIG. 2(d), a semiconductor layer 21 is formed between the plating layer 18 (source electrode) and the plating layer 19 (drain electrode).

The semiconductor layer 21 may be formed, for example, by preparing a solution in which an organic semiconductor material soluble in an organic solvent, such as TIPS pentacene (6,13-bis(triisopropylsilylethynyl) pentacene), is dissolved in the organic solvent, and then applying the solution between the plating layer 18 (source electrode) and the plating layer 19 (drain electrode), and drying it.

In addition, the semiconductor layer 21 may be formed by adding one or more insulating polymers such as polystyrene (PS) or polymethyl methacrylate (PMMA) to the above-described solution, and then applying the solution containing the insulating polymers, and drying it.

When the semiconductor layer 21 is formed in this manner, the insulating polymers are concentrated and formed below the semiconductor layer 21 (on the insulator layer 17 side). When polar groups such as amino groups are present at the interface between the organic semiconductor and the insulator layer, the transistor characteristics tend to deteriorate. However, by providing the organic semiconductor via the insulating polymers described above, deterioration in the transistor characteristics can be suppressed. In this manner, a transistor can be manufactured.

According to the above-described method, it is unnecessary to provide a separate chemical resist or the like in the UV exposure step, and the process can be simplified using only a photomask. Therefore, naturally, a step of removing a resist layer is not required. In addition, due to the catalytic reduction ability of amino groups, a catalyst activation step that is usually required can be omitted, enabling high-precision patterning while achieving significant cost reduction and time savings. In addition, since a dip coating method can be used, it is highly compatible with a roll-to-roll process.

The structure of the transistor is not particularly limited, and can be appropriately selected depending on the purpose. For example, top-contact bottom-gate type, top-contact top-gate type, and bottom-contact top-gate type transistors may be manufactured in the similar manner.

### <Laminate>

The present embodiment is a laminate containing the photosensitive surface treatment agent of the present embodiment.

The laminate of the present embodiment is a laminate in which a substrate and a metal pattern are laminated, and contains a photosensitive surface treatment agent in an unexposed portion where no pattern is formed.

### <Transistor>

The present embodiment is a transistor containing the photosensitive surface treatment agent of the present embodiment.

The laminate of the present embodiment is a transistor having a laminate in which a substrate and a metal pattern are laminated, and contains a photosensitive surface treatment agent in an unexposed portion where no pattern is formed.

### EXAMPLES

Hereinafter, the present invention will be described in more detail using examples, but is not limited to the following examples.

Nitration of 1-(4,5-dimethoxy-2-nitrophenyl)-2-methylpropanone was carried out via the reaction shown below to obtain an intermediate A and an intermediate B.

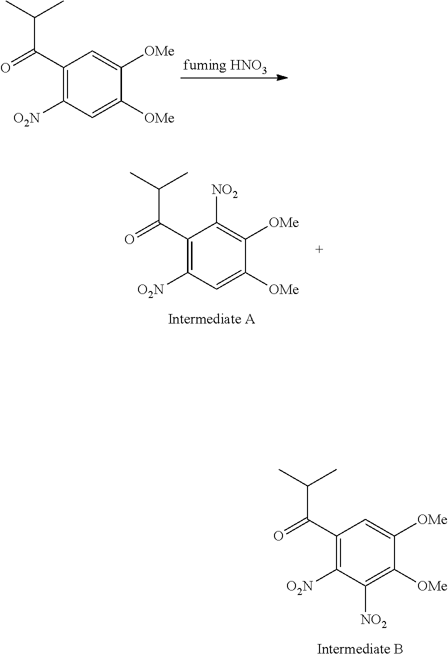 [SMILES: COc1cc(C(=O)C(C)C)c([N+](=O)[O-])cc1OC ; COc1cc([N+](=O)[O-])c(C(=O)C(C)C)c([N+](=O)[O-])c1OC ; COc1cc(C(=O)C(C)C)c([N+](=O)[O-])c([N+](=O)[O-])c1OC]

500 mg (1.97 mmol, 1.0 eq, MI457) of 1-(4,5-dimethoxy-2-nitrophenyl)-2-methylpropanone was placed in a 100 mL wide-mouth eggplant flask, 3 mL (70.2 mol, 36 eq) of fuming HNO₃ was slowly added dropwise at 0° C. (ice water) over 5 minutes, and the mixture was then stirred at room temperature for 30 minutes.

Cold water (45 mL) was added to the reaction solution, which was then stirred at 0° C. for 1 hour. The resultant was then subjected to suction filtration through a membrane filter, rinsed with H₂O (45 mL), and vacuum-dried to obtain 421 mg of a pale brown solid.

The solid was purified by silica gel column chromatography (as a developing solvent, hexane:ethyl acetate=2:1), concentrated, and vacuum-dried to obtain 30 mg (0.10 mmol, 5%) of yellow oil A (intermediate A) as a developing solvent fraction 1, and 144 mg (purity 63%, converted yield 91 mg, 0.30 mmol, 15%) of a pale yellow solid B (intermediate B) as a fraction 2.

Measurement results of the intermediate A and the intermediate B are shown below.

### Intermediate A

¹H NMR (CDCl3) 1.23 (6H, d), 2.82 (1H, m), 4.05 (3H, s), 4.07 (3H, s), 7.81 (1H, s)

¹³C NMR (CDCl3) 18.33, 41.80, 57.14, 62/69, 109.67, 124.30. 140.79, 146.30, 153.48, 201.89

FTIR (NaCl) 1706, 1549, 1341, 1293 cm⁻¹

ESI-MS 321.0730, calcd for C₁₂H₁₄N₂O₇Na [M+Na⁺] 321.0699

Mp. 70.1-72.6° C.

### Intermediate B

¹H NMR (CDCl3) 1.23 (6H, d), 2.92 (1H, m), 4.00 (3H, s), 4.04 (3H, s), 6.87 (1H, s)

¹³C NMR (CDCl3) 18.54, 40.77, 57.24, 62.75, 111.03, 131.26, 134.63, 140.25, 142.46, 157.63, 203.96

ESI-MS 321.0723, calcd for C₁₂H₁₄N₂O₇Na [M+Na⁺] 321.0699

Mp. 115.2-117.2° C.

### Example 1

The intermediate A obtained above was reduced via the reaction shown below to obtain 1-(3.4-dimethoxy-2.6-dinitrophenyl)-2-methylpropan-1-ol.

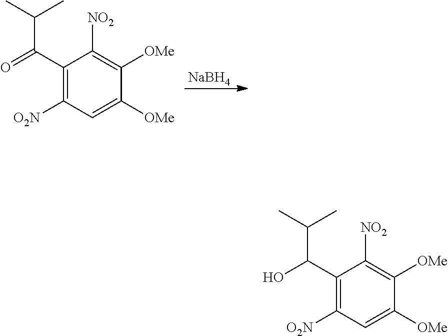 [SMILES: COc1cc([N+](=O)[O-])c(C(=O)C(C)C)c([N+](=O)[O-])c1OC ; "NaBH4" ; COc1cc([N+](=O)[O-])c(C(O)C(C)C)c([N+](=O)[O-])c1OC]

57 mg (0.19 mmol, 1.0 eq) of 1-(3,4-dimethoxy-2,6-dinitrophenyl)-2-methylpropanone was placed in a 20 mL eggplant flask and dissolved in 1 mL of tetrahydrofuran (THF) and 0.5 mL of methanol. 11 mg (0.29 mmol, 1.5 eq) of sodium tetrahydroborate was added little by little at 0° C., and the mixture was stirred at 0° C. for 30 minutes and further stirred at room temperature for 3 hours.

After concentration under reduced pressure, ethyl acetate (2 mL× 5) and water (2 mL) were added for extraction, followed by drying over anhydrous magnesium sulfate, filtration, concentration, and vacuum drying to obtain 46 mg of a brown viscous material. The material was purified by silica gel column chromatography (as a developing solvent, hexane:ethyl acetate=3:1), concentrated, and vacuum-dried to obtain 18 mg (purity 60%, 0.0036 mmol, yield 19%) of yellow oil.

Measurement results of 1-(3.4-dimethoxy-2.6-dinitrophenyl)-2-methylpropan-1-ol are shown below.

¹H NMR (CDCl3) 0.74 (3H, d), 1.08 (3H, d), 2.07 (1H, m), 3.98 (3H, s), 4.00 (3H, s), 4.57 (1H, br), 7.46 (1H, s)

¹³C NMR (CDCl3) 19.26, 19.67, 33.79, 56.81, 62.46, 74.75, 110.26, 123.02, 144.35, 144.54, 145.88, 151.90

FTIR (NaCl) 3565, 2967, 1547, 1350, 1293 cm⁻¹

ESI-MS 323.0883, calcd for C₁₂H16N₂O₇Na [M+Na⁺] 323.0855

Furthermore, a model compound A (1-(3,4-dimethoxy-2,6-dinitrophenyl)-2-methylpropyl N-butylcarbamate) was synthesized via the reaction shown below.

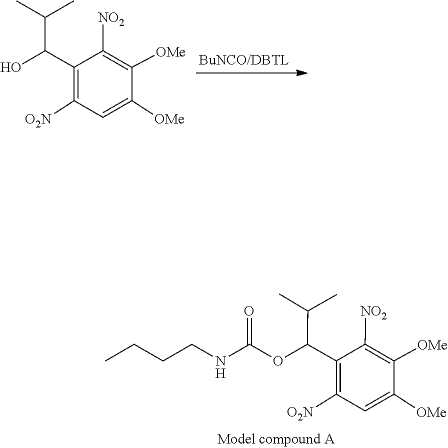 [SMILES: COc1cc([N+](=O)[O-])c(C(O)C(C)C)c([N+](=O)[O-])c1OC ; CCCCNC(=O)OC(c1c([N+](=O)[O-])cc(OC)c(OC)c1[N+](=O)[O-])C(C)C]

17 mg (purity 60%, 0.034 mmol) of 1-(3,4-dimethoxy-2,6-dinitrophenyl)-2-methylpropanol was placed in a 10 mL ground-glass test tube and dissolved in 1 mL of dry tetrahydrofuran (THF), 5 μL (0.008 mmol, 0.2 eq) of dibutyltin dilaurate (DBTL) and 20 μL (0.75 mmol, 22 eq) of butyl isocyanate (BuNCO) were added, and the mixture was refluxed in a nitrogen atmosphere for 19 hours. After concentration under reduced pressure and vacuum drying, 56 mg of a dark brown solid was obtained. The solid was purified by silica gel column chromatography (as a developing solvent, chloroform), concentrated, and vacuum-dried to obtain 13 mg (0.030 mmol, yield 88%) of a target product (model compound A) as yellow oil.

Measurement results of the model compound A are shown below.

¹H NMR (CDCl3) 0.88 (3H, t), 0.89 (3H, d), 0.93 (3H, d), 1.32 (2H, m), 1.47 (2H, m), 2.25

(1H, m), 3.12 (2H, m), 3.97 (6H, s), 4.68 (1H, br), 5.92 (1H, d), 7.52 (1H, s)

¹³C NMR (CDCl3). 13.70, 18.21, 19.80, 19.98, 31.80, 33.18, 40.83, 56.73, 62.42, 74.89, 109.90, 121.37, 144.56, 144.76, 144.83, 151.86, 155.29

ESI-MS 422.1571, calcd for C₁₇H₂₅N₃O₈Na [M+Na⁺] 422.1539

### Example 2

The intermediate B obtained above was reduced via the reaction shown below to obtain 1-(4.5-dimethoxy-2.3-dinitrophenyl)-2-methylpropan-1-ol.

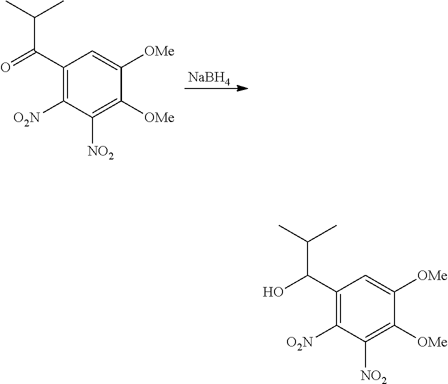 [SMILES: COc1cc(C(=O)C(C)C)c([N+](=O)[O-])c([N+](=O)[O-])c1OC ; "NaBH4" ; COc1cc(C(O)C(C)C)c([N+](=O)[O-])c([N+](=O)[O-])c1OC]

144 mg (purity 63%, conversion 91 mg, 0.30 mmol, 1.0 eq) of 1-(4,5-dimethoxy-2,3-dinitrophenyl)-2-methylpropanone was placed in a 50 mL eggplant flask and dissolved in 2 mL of tetrahydrofuran (THF) and 1 mL of methanol. 17 mg (0.45 mmol, 1.5 eq) of sodium tetrahydroborate was added little by little at 0° C., and the mixture was stirred at 0° C. for 30 minutes and further stirred at room temperature for 3 hours.

After concentration under reduced pressure, ethyl acetate (2 mL× 5) and water (5 mL) were added for extraction, followed by drying over anhydrous magnesium sulfate, filtration, concentration, and vacuum drying to obtain 139 mg of a brown viscous material. The material was purified by silica gel column chromatography (as a developing solvent, hexane:ethyl acetate=2:1), concentrated, and vacuum-dried to obtain 75 mg (0.25 mmol, 82%) of yellow oil as a target product containing the raw materials.

Measurement results of 1-(4.5-dimethoxy-2.3-dinitrophenyl)-2-methylpropan-1-ol are shown below.

¹H NMR (CDCl3) 0.92 (3H, d), 0.97 (3H, d), 1.95 (1H, m), 2.21 (1H, d), 3.99 (3H, s), 4.01 (3H, s), 4.87 (1H, br), 7.31 (1H, s)

¹³C NMR (CDCl3). 16.83, 19.48, 34.70, 56.83, 62.62, 73.28, 111.71, 134.10, 135.89, 140.07, 140.94, 156.12

FTIR (NaCl) 3585, 2964, 1551, 1350, 1291 cm⁻¹

ESI-MS 323.0873, calcd for C₁₂H₁₆N₂O₇Na [M+Na⁺] 323.0855

Furthermore, a model compound B (1-(4,5-dimethoxy-2,3-dinitrophenyl)-2-methylpropyl N-butylcarbamate) was synthesized via the reaction shown below.

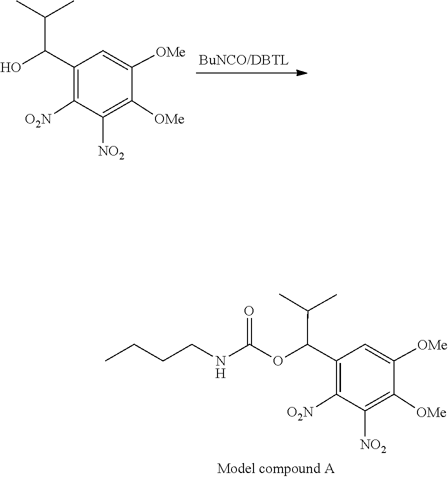 [SMILES: COc1cc(C(O)C(C)C)c([N+](=O)[O-])c([N+](=O)[O-])c1OC ; CCCCNC(=O)OC(c1cc(OC)c(OC)c([N+](=O)[O-])c1[N+](=O)[O-])C(C)C]

150 mg (0.50 mmol) of 1-(4,5-dimethoxy-2,3-dinitrophenyl)-2-methylpropan-1-ol was placed in a 10 mL ground-glass test tube and dissolved in 1.5 mL of dry tetrahydrofuran (THF), 15 μL (0.025 mmol, 0.05 eq) of dibutyltin dilaurate (DBTL) and 85 μL (0.75 mmol, 1.5 eq) of butyl isocyanate (BuNCO) were added, and the mixture was refluxed in a nitrogen atmosphere for 19 hours. After concentration under reduced pressure and vacuum drying, 251 mg of a dark brown solid was obtained. The solid was purified by silica gel column chromatography (as a developing solvent, hexane:ethyl acetate=2:1), concentrated, and vacuum-dried. The resultant was rinsed with hexane and vacuum-dried to obtain 156 mg (0.39 mmol, 78%) of a target product (model compound B) as an orange solid.

Measurement results of the model compound B are shown below.

¹H NMR (CDCl3) 0.90 (3H, t), 0.91 (3H, d), 1.03 (3H, d), 1.32 (2H, m), 1.45 (2H, m), 2.18 (1H, m), 3.13 (2H, m), 3.93 (3H×2, 2s), 4.76 (1H, br), 5.61 (1H, d), 6.97 (1H, s)

¹³C NMR (CDCl3) 13.69, 17.88, 19.21, 19.86, 31.88, 33.39, 40.85, 56.71, 62.58, 76.04, 111.29, 131.17, 134.82, 140.45, 141.14, 155.28, 155.84

Anal. Calcd for C₁₇H₂₅N₃O₈: C 51.12, H 6.31, N 10.52%. Found: C 51.07, H 6.20, N 10.42%

FTIR (KBr disk): 1693, 1557, 1359 cm-1

Mp 106.9-108.1° C.

### Comparative Example 1

A mononitro compound (synthesis of 1-(4,5-dimethoxy-2-nitrophenyl)-2-methylpropyl N-butylcarbamate) was produced via the reaction shown below.

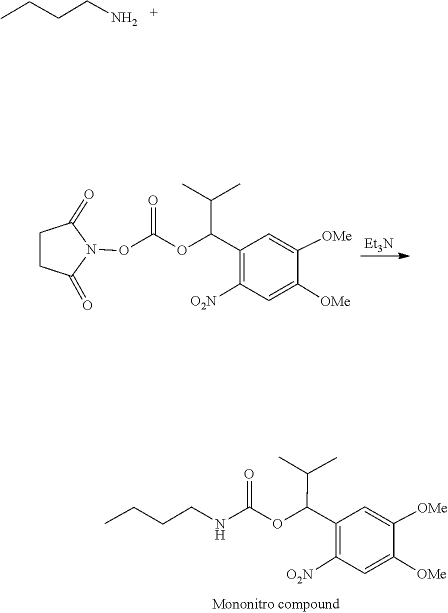

4.0 g (10 mmol) of 1-(4,5-dimethoxy-2-nitrophenyl)-2-methylpropyl (2-methylene-5-oxopyrrolidine-1-yl) carbonate was placed in a 200 mL eggplant flask and dissolved in 40 mL of dry tetrahydrofuran (THF), and 2 mL (20 mmol, 2 eq) of butylamine was added, followed by stirring at room temperature in a nitrogen atmosphere for 3 hours. The resultant was concentrated under reduced pressure, dissolved in 80 mL of ethyl acetate, washed three times with 40 mL of water, dried with anhydrous magnesium sulfate, and vacuum-dried to obtain 4.51 g of a pale yellow solid. The solid was purified by silica gel column chromatography (as a developing solvent, chloroform), concentrated, and vacuum-dried to obtain 3.41 g (9.62 mmol, vield 96%) of a mononitro compound as a target product.

1-(4,5-dimethoxy-2-nitrophenyl)-2-methylpropyl (2-methylene-5-oxopyrrolidine-1-yl) carbonate was synthesized by the previously reported methods (the methods disclosed in Non-Patent Document 1 and Non-Patent Document 2 below).

Non-Patent Document 1: Noriko Chikaraishi Kasuga, Yusuke Saito, Naomichi Okamura, Tatsuya Miyazaki, Hikaru Satou, Kazuhiro Watanabe, Takaki Ohta, Shu-hei Morimoto, Kazuo Yamaguchi, Influences of alpha-substituent in 2-nitrobenzyl-protected esters on both photocleavage rate and subsequent photoreaction of the generated 2-nitrosoketones: A novel photorearrangement of 2-nitrosoketones, J. Photochem. Photobiol. A: Chem., 2016, 321, 41-47

Non-Patent Document 2: Takuma Igari and Kazuo Yamaguchi, 2-Nitrobenzylcarbamate-Bearing Alkylphosphonic Acid Derivative Forms Photodegradable Self-Assembled Monolayer That Enables Fabrication of a Patterned Amine Surface, Chem. Lett. 2017, 46 (8), 1220-1222

Measurement results of the mononitro compound are shown below.

1H-NMR (CDCl3) 0.90 (3H, t), 0.99 (6H, m), 1.31 (2H, sext), 1.46 (2H, quint), 2.14 (1H, sept), 3.14 (2H, m), 3.94 (6H, s), 4.76 (1H, t), 6.23 (1H, d), 6.90 (1H, s), 7.61 (1H, s)

¹³C-NMR (CDCl3) 13.71, 17.20, 19.43, 19.88, 32.04, 33.34, 40.78, 56.33, 75.74, 107.97, 108.94, 132.17, 140.65, 147.76, 153.02, 155.73

FTIR (KBr) 3303, 1685, 1518, 1274 cm⁻¹

<Measurement of Photodecomposition Rate Constant k (s⁻¹)>

The above-obtained model compound A, model compound B and mononitro compound were each dissolved in acetonitrile to prepare 0.1 mM solutions.

The solutions were irradiated with light having a wavelength of 365 nm and an illuminance of 25 mW/cm² from an ultra-high-pressure mercury lamp through a 365 nm bandpass filter and a water filter for 5, 10, 15, 20, 25, and 30 seconds, and then subjected to HPLC measurement.

The peak surface areas of the raw materials obtained by HPLC measurement (S₀: area before light irradiation, St: area t seconds after light irradiation) were substituted into the following equation to determine the photodecomposition rate constant k (s⁻¹) based on the reduction rate of the raw materials. The results are shown in Table 1.

$$ln ( \frac{{S}_{t}}{{S}_{0}} ) = - kt$$

<Measurement of molar absorption coefficient (8365/M⁻¹ cm⁻¹)

The above-obtained model compound A, model compound B and mononitro compound were each dissolved in acetonitrile to prepare 0.1 mM solutions. Each solution was placed in a quartz cell with an optical path length of 1 cm, and the molar absorption coefficient (ε₃₆₅/M⁻¹ cm⁻¹) was calculated from the absorbance measured using a UV-visible spectrophotometer (V-570, manufactured by JASCO Corporation).

<Calculation of Half-Life (t_{1/2}s)>

The half-life (t_{1/2}s) was calculated from the photodecomposition rate constant.

### <Calculation of Photoreaction Efficiency (Φ365)>

The photoreaction efficiency (Φ365) was calculated by the following method from the photodecomposition rate constant and absorbance (A365) measured using the solution with a concentration of 0.1 mM.

$$Photoreaction efficiency ( Φ365 ) = photodecomposition rate constant ( k ) / absorbance ( A 365 )$$

TABLE 1

| | | | Photo- | | Photo- |
|---|---|---|---|---|---|
| | | Molar ab- | decom- | | re- |
| | | sorption | position | | action |
| | | coefficient | rate | Half- | efficien- |
| | | ε₃₆₅/ | constant | life | cy |
| | | M⁻¹ cm⁻¹ | k (s⁻¹) | t_{1/2}/s | Φ₃₆₅ |
| Example 1 | Model compound A | 1,000 | 0.049 | 14 | 0.49 |
| Example 2 | Model compound B | 2,300 | 0.100 | 7 | 0.43 |
| Comparative | Mononitro | 4,100 | 0.046 | 15 | 0.11 |
| Example 1 | compound | | | | |

It was confirmed that the model compounds A and B having a dinitrobenzyl group have about four times or more higher photoreaction efficiency than the mononitro compound having a mononitrobenzyl group.

In addition, the model compounds A and B having a dinitrobenzyl group had a faster photodecomposition rate than the mononitro compound having a mononitrobenzyl group.

Furthermore, the comparison between the model compounds A and B confirmed that the model compound B had a faster photodecomposition rate.

Based on the results of the above-described model compounds A and B, it can be sufficiently inferred that the compound represented by Formula (M1), which has a dinitrobenzyl group, and the polymer compound represented by Formula (P1) also exhibit similar effects of high photoreaction efficiency and an increased photodecomposition rate.

### REFERENCE SIGNS LIST

11 Substrate
10a Photosensitive surface treatment agent
10 Photosensitive surface treatment agent layer
13 Photomask
14 Amine-generated portion
12 Amine-non-generated portion
15 Catalyst layer
16 Plating layer
17 Insulator layer
18 Plating layer (source electrode)
19 Plating layer (drain electrode).
21 Semiconductor layer

1. A compound represented by General Formula (M1) below 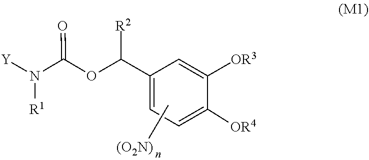
(in Formula (M1), Y is a linear or branched alkyl group having 1 to 10 carbon atoms, a polymerizable group-containing group, or a group represented by [SiX₃—Y¹¹—*], Y¹¹ is a linear or branched alkylene group having 1 to 4 carbon atoms, X is a halogen atom or an alkoxy group, * is a bonding site to an N atom, R¹ is a hydrogen atom or a methyl group, R² is a hydrogen atom or an alkyl group having 1 to 6 carbon atoms, R³ and R⁴ each independently represents an alkyl group having 1 to 3 carbon atoms or a fluoroalkyl group having 1 to 3 carbon atoms, and n=2).

2. A polymer compound having a repeating unit represented by General Formula (P1) below 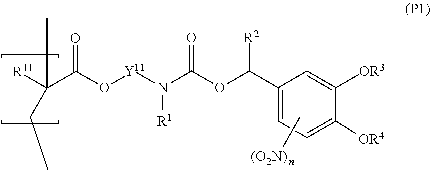
(in Formula (P1), R¹¹ is a hydrogen atom or a methyl group, and Y¹¹ is a linear or branched alkylene group having 1 to 4 carbon atoms, R¹ is a hydrogen atom or a methyl group, R² is a hydrogen atom or an alkyl group having 1 to 6 carbon atoms, R³ and R⁴ each independently represents an alkyl group having 1 to 3 carbon atoms or a fluoroalkyl group having 1 to 3 carbon atoms, and n=2).

3. A photosensitive surface treatment agent, comprising:
the compound according to claim 1 or the polymer compound according to claim 2.

4. A laminate comprising:
the photosensitive surface treatment agent according to claim 3.

5. A substrate for pattern formation having a surface chemically modified using the photosensitive surface treatment agent according to claim 3.

6. A transistor comprising:
the photosensitive surface treatment agent according to claim 3.

7. A pattern formation method, comprising:
a step of applying the photosensitive surface treatment agent according to claim 3 on a substrate to form a photosensitive resin film;
a step of irradiating the photosensitive resin film with light having a predetermined pattern; and
a step of performing electroless plating on at least part of region irradiated with light having the predetermined pattern.

8. A pattern formation method, comprising:
a step of applying the photosensitive surface treatment agent according to claim 3 on a substrate to form a photosensitive resin film;
a step of irradiating the photosensitive resin film with light having a predetermined pattern; and
a step of placing an electroless plating catalyst in at least part of region irradiated with light having the predetermined pattern to perform electroless plating.

9. A pattern formation method, comprising:
a step of applying the photosensitive surface treatment agent according to claim 3 on a substrate to form a photosensitive resin film;
a step of forming an amine generation region in an exposed area of the photosensitive resin film irradiated with light having a predetermined pattern; and
a step of placing an electroless plating catalyst in the amine generation region to perform electroless plating.

10. A transistor manufacturing method, comprising:
a step of forming at least one electrode selected from a source electrode, a drain electrode, or a gate electrode by the pattern formation method according to claim 7.

11. A transistor comprising:
a compound represented by Formula (M1) below or a polymer compound having a repeating unit represented by Formula (P1) below 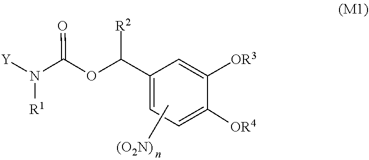
(in Formula (M1), Y is a linear or branched alkyl group having 1 to 10 carbon atoms, a polymerizable group-containing group having 1 to 10 carbon atoms, or a group represented by [SiX₃—Y¹¹—*], Y¹¹ is a linear or branched alkylene group having 1 to 4 carbon atoms, X is a halogen atom or an alkoxy group, * is a bonding site to an N atom, R¹ is a hydrogen atom or a methyl group, R² is a hydrogen atom or an alkyl group having 1 to 6 carbon atoms, R³ and R⁴ each independently represents an alkyl group having 1 to 3 carbon atoms or a fluoroalkyl group having 1 to 3 carbon atoms, and n=2) 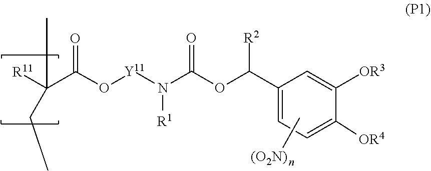
(in Formula (P1), R¹¹ is a hydrogen atom or a methyl group, and Y¹¹ is a linear or branched alkylene group having 1 to 4 carbon atoms, R¹ is a hydrogen atom or a methyl group, R² is a hydrogen atom or an alkyl group having 1 to 6 carbon atoms, R³ and R⁴ each independently represents an alkyl group having 1 to 3 carbon atoms or a fluoroalkyl group having 1 to 3 carbon atoms, and n=2).

12. The transistor according to claim 11,
wherein the compound or the polymer compound has a moiety where at least some nitrobenzyl groups are eliminated, generating amino groups.
